(19) United States
(12) Patent Application Publication
(10) Pub. No.: US 2019/0274887 A1
(43) Pub. Date: Sep. 12, 2019

(54) METHODS, DEVICES AND SYSTEMS FOR TREATING EUSTACHIAN TUBE DISORDERS

(71) Applicant: MEDTRONIC XOMED, INC., Jacksonville, FL (US)

(72) Inventor: Kyle A. Godwin, Costa Mesa, CA (US)

(73) Assignee: MEDTRONIC XOMED, INC., Jacksonville, FL (US)

(21) Appl. No.: 16/299,563

(22) Filed: Mar. 12, 2019

Related U.S. Application Data
(60) Provisional application No. 62/647,025, filed on Mar. 23, 2018.
(60) Provisional application No. 62/641,740, filed on Mar. 12, 2018.

(51) Int. Cl. A61F 11/00; A61B 1/00; A61B 1/015; A61B 1/233; A61M 29/02

(52) U.S. Cl. CPC A61F 11/004; A61B 1/00119; A61B 1/00135; A61M 2210/0675; A61B 1/015; A61B 1/233; A61M 29/02; A61B 1/00094

(57) ABSTRACT

A device for dilating an Eustachian tube of a patient including an inner member, an inflation fluid supply tube, a balloon, and a coupler. The inner member includes a lumen fluidly open at distal and proximal ends. The inner member has a distal portion, a proximal portion, and a central portion. The inflation fluid supply tube is fixedly disposed around the central. The inflation fluid supply tube has an opening disposed between first and second ends. A proximal portion of the inner member extends through the opening and the opening is fluidly sealed against an exterior surface of the inner member. The coupler is configured to selectively couple the inflation fluid supply tube to an endoscope. The coupler is configured to engage against an exterior surface of the inflation fluid supply tube. The coupler is disposable between the first end and the opening of the inflation fluid supply tube.

### CROSS-REFERENCE TO RELATED APPLICATIONS

This Non-Provisional Patent Application claims the benefit of the filing dates of U.S. Provisional Patent Application Ser. No. 62/647,025, filed Mar. 23, 2018, entitled “Therapy Delivery System,” and U.S. Provisional Patent Application Ser. No. 62/641,740, filed Mar. 12, 2018, entitled “Methods, Devices and Systems for Treating Eustachian Tube Disorders,” the entire teachings of which are incorporated herein by reference.

### BACKGROUND

Certain medical treatment devices access the inside of the body through minimally invasive techniques. For example, dilation catheter devices can be introduced into an anatomical passageway in a patient for dilation and treatment. The catheter device may be used to dilate anatomical structures within the ear, nose or throat of a human or animal subject. This may include dilation of ostia of paranasal sinuses (e.g., to treat sinusitis), dilation of the larynx, dilation of the Eustachian tube, dilation of other passageways within the ear, nose, or throat, etc. The catheter and treatment device utilized depends upon the medical procedure performed, the anatomical delivery route, and the individual patient's anatomy, among other factors. Physicians who perform procedures using these devices rely in part upon experience and the known anatomy for appropriate placement. However, the accuracy and ease of placement can be greatly enhanced by using an endoscope with the device.

Visibility of both the anatomy and the surgical instrument are critical during surgeries of the ear, nose, and throat (ENT), for example. Viewing endoscopes can permit visualizing remote internal surgical locations within a patient at targeted areas by accessing those locations through a natural body lumen during ENT surgeries. Endoscopes permit remote visualization within the anatomical passageway (e.g., the ear, nose, throat, paranasal sinuses, etc.) to position a balloon dilator, for example, at desired locations while a surgical procedure is being performed.

The Eustachian tube, sometimes referred to as the auditory tube or the pharyngotympanic tube, is a tube that connects the tympanic cavity of the middle ear to the nasopharynx. The Eustachian tube acts as a pressure equalizing tube from the lateral nasopharynx to the middle ear. At the nasopharynx, the Eustachian tube is bounded by the torus of the Eustachian tube and forms the pharyngeal opening of the Eustachian tube (also known as the pharyngeal ostium).

The Eustachian tube includes a cartilaginous portion and an osseous (bone) portion. There are several muscles that affect the function of the Eustachian tube, including muscles of the soft palate (e.g., the levator veli palatini and tensor veli palatini) and muscles of the ear (e.g., tensor tympani).

Eustachian tube dysfunction (ETD) is a common problem for both children and adults. ETD can be caused by inflammation of the tissue of or near the Eustachian tube, for example. ETD may result in Eustachian tube blockage and/or cause the Eustachian tube to resist opening. When the Eustachian tube is obstructed either through anatomical or inflammatory reasons, the middle ear is not able to equalize pressure which can lead to negative pressure and fluid build-up or retraction of the tympanic membrane.

Advancing and precisely positioning a therapy device within the Eustachian tube for treatment of ETD can be difficult. A device that is too rigid can damage the anatomy of the patient during advancement due to inflexibility, while a device that is too flexible can be difficult to advance through torturous passages. Physicians who perform procedures using these devices rely in part upon experience and the known anatomy for appropriate placement. Endoscopes can provide internal visualization, but the precise location of the endoscope within the body may not always be clear to the physician throughout the procedure, making a navigation attachment useful, particularly during delicate or complex procedures.

Surgeon's typically use one hand to hold and manipulate the endoscope and the other hand to independently and separately hold and manipulate the surgical device. Although an endoscope can assist with remote treatment capability by permitting direct visualization of the treatment site, the patient's anatomy may be too small to provide ease of independent viewing with the endoscope and manipulation of a surgical device, such as a dilation catheter device, for example, within the available space. Combining the endoscope and surgical device can be difficult due to the various manufacturers producing endoscopes and accessories of differing diameters. Mounting a particular surgical treatment instrument, such as a dilation catheter device, and an endoscope together can be problematic due to varying sizes and other factors.

### SUMMARY

The inventors of the present disclosure recognized that a need exists for devices, systems and methods that address one or more of the above-mentioned problems. For example, an endoscope and operative treatment device that can be attached and operated together within the patient's anatomy to avoid the problems encountered with separate devices.

Some aspects of the present disclosure relate to a dilation treatment device for use in dilating an Eustachian tube of a patient. The device includes an inner member, an inflation fluid supply tube, a balloon, and a coupler. The inner member includes an elongated tubular body and defines a lumen fluidly open at a distal end and a proximal end. The inner member has a distal portion terminating at the distal end. A proximal portion terminates at the proximal end. A central portion extends between the distal portion and the proximal portion. The inflation fluid supply tube is fixedly disposed around the central portion of the inner member and includes an inflation lumen. The inflation fluid supply tube has a first end, a second end opposite the first end, and an opening disposed between the first end and the second end. The proximal portion of the inner member extends through the opening and the opening is fluidly sealed against an exterior surface of the inner member. The distal portion of the inner member extends distal the first end. The balloon has a distal side opposite a proximal side. The distal side of the balloon is coupled to the inner member adjacent the distal end. The proximal side of the balloon is in fluid communication with the inflation lumen. The coupler is configured to selectively couple the inflation fluid supply tube to an endoscope. The coupler is configured to engage against an exterior surface of the inflation fluid supply tube. The coupler is disposable between the first end and the opening of the inflation fluid supply tube.

Other aspects of the present disclosure relate to a dilation treatment system including a dilation treatment device, an endoscope, and one or more couplers. The dilation treatment device includes an inner member, an inflation fluid supply tube, and an endoscope. The inner member includes an elongated tubular body and defines a lumen. The lumen is fluidly open at and between a distal end and a proximal end. The inflation fluid supply tube is fixedly disposed around a central portion of the inner member. The inflation fluid supply tube includes a first end, a second end, and an inflation lumen extending between the first end and the second end. The distal end of the inner member extends distally of the first end. The proximal end of the inner member extends through an opening of the inflation fluid supply tube. The balloon is mounted to a distal portion of the inner member and is fluidly coupled to the inflation lumen. The endoscope includes an endoscopic insertion unit defining a viewing end. The coupler is configured to selectively couple the endoscopic insertion unit to the dilation treatment device with the endoscopic insertion unit extending parallel and longitudinally fixed relative to the inflation fluid supply tube. The viewing end is disposed alongside a distal section of the inflation fluid supply tube. The coupler is disposed between the first end and the opening of the inflation fluid supply tube.

Yet other aspects of the present disclosure relate to a method of endoscopically treating an Eustachian tube of a patient. The method includes coupling a dilation treatment device and an endoscope together with one or more couplers to form a dilation treatment system. The dilation treatment device includes an inner member fixedly extending within an inflation fluid supply tube. The inner member includes a distal portion extending distal of a first end of the inflation fluid supply tube and a proximal portion extending proximally outside of the inflation fluid supply tube. A balloon is disposed on the distal portion of the inner member and is fluidly coupled to the inflation fluid supply tube. The endoscope includes an endoscopic insertion unit defining a viewing end. The step of coupling includes the dilation treatment device and the endoscopic insertion unit longitudinally fixedly aligned and coupled together with the one or more couplers, and the viewing end fixedly disposed proximal to the first end of the inflation fluid supply tube and proximal to the distal portion. The method also includes advancing the dilation treatment system into a patient's anatomy. A position of the distal portion of the inner member within the body lumen is visualized with the endoscope. The distal portion of the inner member is positioned at a target site within the patient's Eustachian tube. The balloon is inflated through the inflation fluid supply tube at the target site. An opening of the patient's Eustachian tube at the target site is dilated with the inflated balloon.

### BRIEF DESCRIPTION OF THE DRAWINGS

FIG. 1 is a side view of a dilation treatment system in accordance with principles of the present disclosure.

FIG. 2 is an enlarged, longitudinal cross-sectional view of a portion of the dilation treatment system of FIG. 1.

FIG. 3A is an enlarged, lateral cross-sectional view of a coupler in accordance with principles of the present disclosure and useful with the dilation treatment system of FIG. 1.

FIG. 3B is a side view of the coupler of FIG. 3A.

FIG. 3C is an enlarged cross-sectional view of the dilation treatment system of FIG. 1, taken along the line A-A.

FIG. 4 is an exploded side view of the dilation treatment system of FIG. 1.

### DETAILED DESCRIPTION

Surgical devices and systems embodying principles of the present disclosure can be employed in various types of surgical procedures including, but not limited to, treatment of Eustachian tube dysfunction. The surgical treatment systems, in accordance with aspects of the present disclosure, can provide for ease of use to the surgeon by allowing the surgeon to operate both an endoscope and a balloon dilation device simultaneously with one hand. Additionally, attaching the endoscope and balloon dilation devices together allows the surgeon to simultaneously insert and efficiently manipulate and operate the devices through the limited space available to treat the Eustachian tube.

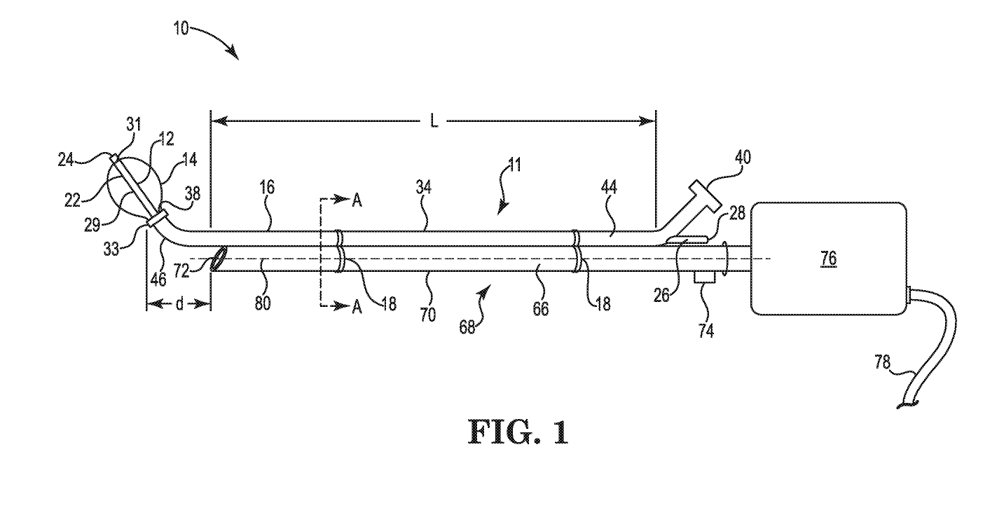

FIG. 1 illustrates a side view of dilation treatment system 10 in accordance with aspects of the present disclosure. The dilation treatment system 10 includes a dilation treatment device 11 having an inner member 12, a balloon 14, and an inflation fluid supply tube 16. In general terms, the balloon 14 is mounted on the inner member 12. The inflation fluid supply tube 16 is disposed around the inner member 12 and is fluidly coupled to the balloon 14. The dilation treatment system 10 also includes a coupler 18. The coupler 18 can be configured to couple the dilation treatment device 11 to an endoscope 20. Details on the various components are provided below. In general terms, however, the endoscope 20 is coupled to, and extends with, the inflation fluid supply tube 16 for simultaneous visualization and positioning of the balloon 14 at a target site such as for treating a patient's Eustachian tube.

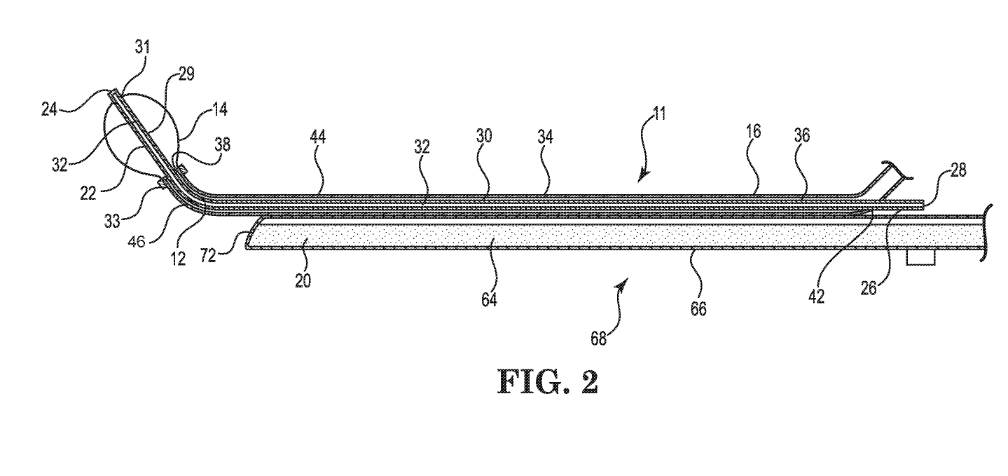

With additional reference to FIG. 2, the inner member 12 has a distal portion 22 terminating at a distal end 24, a proximal portion 26 terminating at a proximal end 28, and a center portion 30 extending between the distal portion 22 and the proximal portion 26. The distal portion 22 can be shaped by a user or otherwise formed in a predetermined shape. In some non-limiting embodiments, the distal portion 22 is shaped to include or define a bend that is 55°+/−10°. The distal portion 22 can be bendable by the user without breaking or losing required strength to assume a shape useful in placement of at least the distal end 24 in the specific anatomy of a patient, and to retain substantially that shape during a dilation procedure. In one embodiment, the distal portion is 25 mm to 30 mm in length. The distal end 24 is sized and shaped to be insertable into a patient's nostril and advancing the dilation treatment device 11 within the Eustachian tube.

The inner member 12 includes an exterior surface 29 and defines a lumen 32. The lumen 32 is fluidly open at and between the distal end 24 and the proximal end 28. The lumen 32 of the inner member 12 is suitable for pressure relief, irrigation, or aspiration. In some non-limiting embodiments, the inner member 12 can have an outer diameter on the order of 1 mm. The inner member 12 has a length suitable to extend fully within the patient's anatomy to the target site to position the distal portion 22 at the target site while the proximal portion 26 extends outside of the patient. The proximal end 28 can be positioned exterior of the patient to be fluidly open to the atmosphere or connectable to an aspiration source or an irrigation source, as appropriate. In some embodiments, the proximal portion 26 can also provide for grasping by a user or robotic arm from exterior of the patient.

The inner member 12, along with one or more other components of the dilation treatment device 11, is formed of biocompatible materials. Additionally, all or portions of the inner member 12 can be formed of a malleable material. The inner member 12 is formed to be manually bendable to a desired shape and to substantially retain the shape during a dilation procedure. For example, the center portion 30 and the proximal portion 28 of the inner member 12 can be malleable in conjunction with, or separate from, the distal portion 22. As used herein, the term “malleable” refers to being cable of being shaped, bent, or otherwise deformed by forces produced by manual manipulation of a human user such that the malleable element retains the deformation. The materials can be malleable, bendable, flexible and combinations of these characteristics. The inner member 12 can be formed of medical grade annealed aluminum, stainless steel, or nitinol, for example.

The balloon 14 can be an expandable balloon that is transitionable between a low-profile contracted, deflated state against the inner member 12 and an expanded, inflated state. The balloon 14 is illustrated in the expanded state. In some embodiments, the balloon 14 is sized and shaped for dilating a patient's Eustachian tube when transitioned to or toward the expanded state. The balloon 14 has a distal side 31 and a proximal side 33. In one embodiment, the balloon 14 is approximately 20 mm in length (between the distal and proximal side 31, 33) and has an expanded diameter of 5 mm to 7 mm. In some embodiments, the balloon 14 can be transparent or translucent. The balloon 14 is formed of a flexible biocompatible material. Any suitable expandable medical balloon material and construction available may be used.

The inflation fluid supply tube 16 is an elongate tubular sheath having an exterior surface 34. The inflation fluid supply tube 16 is sized to fit around the inner member 12 and includes or defines an inflation lumen 36 extending therethrough between opposing first and second ends 38, 40. An opening 42 is disposed between the first end 38 and the second end 40 of the inflation fluid supply tube 16. In one embodiment, the opening 42 is an aperture in a sidewall 44 of the inflation fluid supply tube 16. A distal section 46, terminating at the first end 38, can be shaped by a user or otherwise formed in a predetermined shape. In one embodiment, at least the distal section 46 of the inflation fluid supply tube 16 can be formed of a malleable material. The distal section 46 can be bendable by the user without breaking or losing required strength to assume a different shape useful in placement in the specific anatomy of a patient, and to retain substantially that shape during a dilation procedure. The inflation fluid supply tube 16 is formed of a material that does not substantially change in length during pressurization, such as pressurization used to inflate the balloon 14. The inflation fluid supply tube 16 substantially maintains a length between the first and second ends 38, 40 during pressurization of the balloon 14 and when not pressurized. The inflation fluid supply tube 16 is formed of a material that is suitably rigid to maintain the inflation lumen 36 along the length proximal of the distal section 46.

When assembled, the inner member 12 is longitudinally fixed within the inflation fluid supply tube 16. More particularly, the center portion 30 of the inner member 12 is disposed within the inflation fluid supply tube 16. The center portion 30 of the inner member 12 can extend within the inflation lumen 36 of the inflation fluid supply tube 16 or can be fluidly separated from the inflation lumen 36 (not shown). In one embodiment, the inflation fluid supply tube 16 is heat welded onto the inner member 12. In one embodiment, the inner member 12 is adhered to the inflation fluid supply tube 16 at the first end 38. In another embodiment, the inner member 12 is adhered to the inflation fluid supply tube 16 within the inflation fluid supply tube 16.

The distal portion 22 and proximal portion 26 of the inner member 12 extend and terminate outside of the inflation fluid supply tube 16. The proximal portion 26 of the inner member 12 extends through the opening 42 in the sidewall 44 of the inflation fluid supply tube 16. The opening 42 is configured to provide sealed passage of the inner member 12 from an interior to an exterior of the inflation fluid supply tube 16. The inflation fluid supply tube 16 can be fluidly sealed around the exterior surface 29 of the inner member 12 at the opening 42. For example, the opening 42 can be sealed against the inner member 12 with an interference fit, an adhesive or knurled. Other suitable means of sealing are also acceptable. The opening 42 is positioned along the inflation fluid supply tube 16 such that the proximal portion 26 of the inner member 12 extends a suitable distance from the first end 38 so as to be located at the exterior of the patient when the balloon dilation device 11 is inserted into an insertion pathway of the patient to the target treatment site.

The distal portion 22 of the inner member 12 extends distally through and at least partially beyond the first end 38 of the inflation fluid supply tube 16. The distal portion 22 can extend partially within the distal section 46 of the inflation fluid supply tube 16. The distal portion 22 of the inner member 12 extends beyond, or distal to, the first end 38 of the inflation fluid supply tube 16 to maintain the balloon 14. The balloon 14 is disposed around the inner member 12 with the distal side 31 of the balloon 14 coupled to the distal portion 22 of the inner member 12. The distal portion 22, along with the distal section 46 of the inflation fluid supply tube 16, provides suitable flexibility to the balloon dilation device 11 and can be bent to a desired shape to accommodate the patient's anatomy. In one embodiment, the inner member 12 and/or the inflation fluid supply tube 16 can be formed into a pre-determined geometry during manufacturing. In another embodiment, the inner member 12 and/or the inflation fluid supply tube 16 are formed to a determined shape by the surgeon or user.

The balloon 14 is mounted adjacent the distal end 24 of the inner member 12 along the distal portion 22 with the proximal side 33 of the balloon 14 coupled to the inflation fluid supply tube 16. The balloon 14 can be fixedly coupled to the inner member 12 and the inflation fluid supply tube 16 by welding, adhesive, or other suitable means. In one embodiment, the balloon 14 is disposed along the distal portion 22 with the distal side 31 disposed a suitable distance from the terminal distal end 24 of the inner member 12 to seal and couple the balloon 14 against the inner member 12 and maintain the internal pressure of the balloon 14 when inflated to the expanded state. In one embodiment, the distal side 31 of the balloon 14 is positioned 3 millimeters to 4 millimeters (mm) from the distal end 24 of the inner member 12. The first end 38 of the inflation fluid supply tube 16, and in particular, the inflation lumen 48, fluidly communicates with and terminates at the balloon 14 to inflate, or expand, the balloon 14, for example. The inflation fluid supply tube 16 can fluidly couple the inflation lumen 36 at the first end 38 of the inflation fluid supply tube 16 to the balloon 14 for rapid inflation and deflation of the balloon 14. The inflation fluid supply tube 16 does not expand under the pressures used to expand the balloon 14.

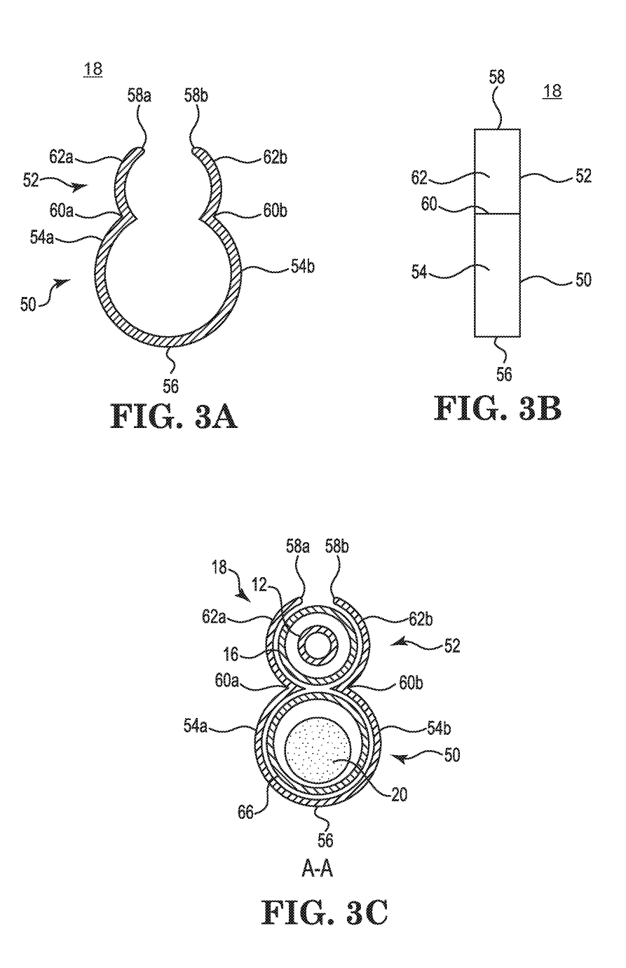

The coupler 18 can be employed to attach the balloon dilation device 11 to the endoscope 20. FIGS. 3A and 3B illustrate enlarged front and side views of the coupler 18 useful with the dilation treatment system 10 in accordance with aspects of the present disclosure. With particular reference to FIGS. 3A and 3B, in one embodiment, the coupler 18 can be formed in an open “8” shape including a first portion 50 and a second portion 52 configured to accommodate and secure the balloon dilation device 11 and the endoscope 20 together. In one embodiment, the coupler 18 can include opposing legs 54a, 54b joined together at a base 56 and terminating at ends 58a, 58b. The legs 54a, 54b are biased toward one another to a securing position. The first portion 50 can be defined by legs 54a, 54b extending from the semi-circular shaped base 56 to joinders 60a, 60b, respectively. The second portion 52 can include the portion of legs 54a, 54b extending from the joinders 60a, 60b to opposing inwardly curved feet 62a, 62b and terminating at the ends 58a, 58b, respectively. Other shapes of the coupler 18 are also acceptable. The coupler 18 is formed of a biocompatible material that is suitably flexible. The coupler 18 is formed of a material that can conform to endoscopes of varying diameters. The coupler 18 can be formed of a soft plastic, aluminum, or other malleable material, for example.

Returning to FIGS. 1 and 2, the endoscope 20 can be coupled to the balloon dilation device 11 using one or more of the couplers 18 to form the dilation treatment system 10. The endoscope 20 is capable of assisting with properly positioning the balloon 14 of the balloon dilation device 11. The endoscope 20 can be of a conventional design and includes a guide body, or insertion body 64, within which appropriate illumination and imaging components are maintained (not shown). A sheath 66 can optionally be provided over the insertion body 64. Regardless, an endoscopic insertion unit 68 having an exterior surface 70 is provided that, in some embodiments, includes the insertion body 64 and the sheath 66, and in other embodiments, does not include the sheath 66.

The insertion body 64, as well as the sheath 66 when provided, can be flexible in order to traverse the patient's anatomy without damage to the anatomy. The insertion body 64 can be a variety of sizes (e.g., length and diameter) suitable for insertion and navigation within the patient's anatomy. The insertion body 64 terminates at a viewing end 72. The viewing end 72 can be angled, rounded or squared off. The sheath 66 is sized and shaped to maintain the insertion body 64 and facilitate the provision of irrigation or suctioning to remove surgical debris from the viewing end 72 and can include air tubes, water tubes, or suction tubes (not shown) to flush away or suction away surgical debris from the viewing end 72. The sheath 66 can include a connection assembly 74 as an inlet and fluid connection to an irrigation or aspiration source (not shown). The endoscope 20 can include a camera 76 connectable to a monitor by way of a camera cable 78. Images received through the endoscope 20 can be view on the monitor by a user.

With additional reference to FIG. 3C, the coupler 18 can be selectively disposed along a length of the endoscopic insertion unit 68 to selectively couple the endoscope 20 to the balloon dilation device 11 to form the dilation treatment system 10. More particularly, one or more couplers 18 can be employed to couple the endoscopic insertion unit 68 to the inflation fluid supply tube 16. The one or more coupler 18 can be circumferentially disposable around the inflation fluid supply tube 16 and the endoscopic insertion unit 68. In one embodiment, the first portion 50 can be sized to be circumferentially disposed around and engage the exterior surface 70 of the endoscopic insertion unit 68, for example. In one embodiment, the first portion 50 is suitably flexible to accommodate variously sized endoscopic insertion units 68. The curvature and length of the feet 62a, 62b of the second portion 52 can similarly be disposed around and engage with the exterior surface 34 of the inflation fluid supply tube 16, for example. Alternatively, the first portion 50 accommodates the inflation fluid supply tube 16 and the second portion 52 accommodates the endoscopic insertion unit 68. Regardless, the coupler 18 can frictionally engage against the exterior surface 34 of the inflation fluid supply tube 16 and the exterior surface 70 of the endoscopic insertion unit 68.

With reference to FIG. 1, the one or more couplers 18 can be disposed in a spaced relationship to another along a commonly shared adjoining length “L” of the inflation fluid supply tube 16 and the endoscopic insertion unit 68. The coupler 18 can be inserted over and around the endoscopic insertion unit 68 and the inflation fluid supply tube 16 in a direction perpendicular to a longitudinal axis 80 of the endoscopic insertion unit 68. The coupler 18 can be adaptable to accommodate and be disposed circumferentially around variously circumferentially sized endoscopic insertion units 68. In some embodiments, the coupler 18 can be removably coupled to the endoscopic insertion unit 68 and the inflation fluid supply tube 16. In some embodiments, the coupler 18 can be non-removably, or permanently, attached to at least one of the endoscopic insertion unit 68 and the inflation fluid supply tube 16.

With continued reference to FIGS. 1 and 2, in some embodiments, the endoscopic insertion unit 68 and the inflation fluid supply tube 16 maintaining the inner member 12, are coupled together by the coupler 18 such that the inflation fluid supply tube 16 and the endoscopic insertion unit 68 are adjoining and extending longitudinally in parallel together along the endoscopic insertion unit 68. In one embodiment, the coupler 18 maintains the inflation fluid supply tube 16 in a secured longitudinally fixed relationship to the endoscopic insertion unit 68. In any regard, the coupler 18 secures the balloon dilation device 11 and endoscope 20 together in a longitudinally joined relationship. By coupling the endoscope 20 and the balloon dilation device 11, the dilation treatment system 10 employs a medical treatment device 11 that is combined with the endoscope 20 as a complete dilation treatment system 10 capable of efficiently and accurately positioning the balloon 14 for dilation of the Eustachian tube.

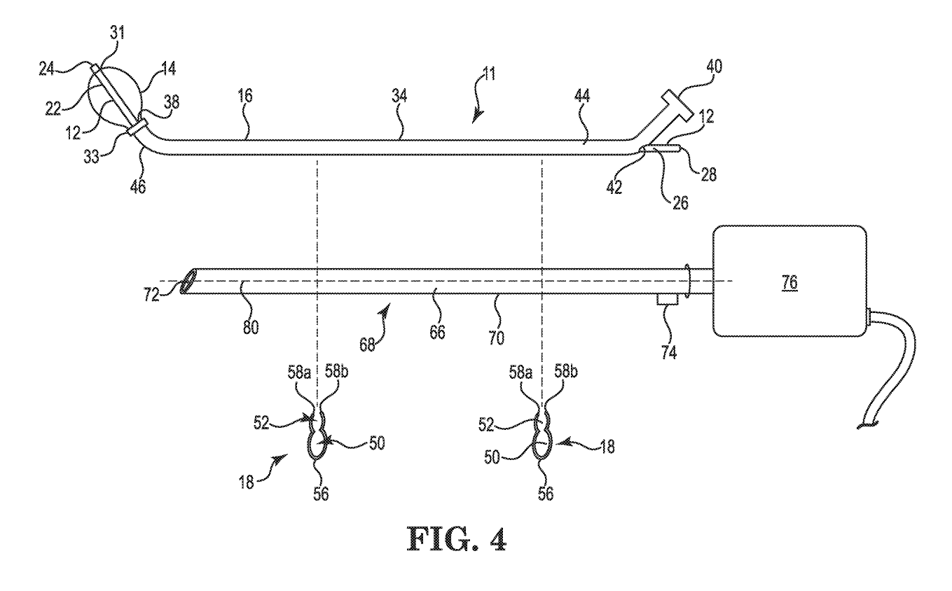

With additional reference to the exploded view of FIG. 4, to couple, or adjoin, the inflation fluid supply tube 16 and the endoscopic insertion unit 68, the coupler 18 can be slid over the endoscopic insertion unit 68 and the inflation fluid supply tube 16 in a direction perpendicular to the longitudinal axis 80 to engage with the exterior surfaces 34, 70 of the inflation fluid supply tube 16 and the endoscopic insertion unit 68, respectively. More particularly, the endoscopic insertion unit 68 can first be inserted between opposing ends 58a, 58b of legs 54a, 54b (FIG. 3A) and then between the gap between the joinders 60a, 60b (FIG. 3A) with the diameter of the endoscopic insertion unit 68 forcing the legs 54a, 54b to temporarily extend apart until the endoscopic insertion unit 68 is fully inserted into the first portion 50. Once so-positioned, the coupler 18 self-reverts to a normal state, with the legs 54a, 54b frictionally engaging the endoscopic insertion unit 68. The inflation fluid supply tube 16 can then be similarly inserted into the second portion 52. Once so-positioned, the coupler 18 self-reverts to a normal state, with the legs 54a, 54b frictionally engaging the inflation fluid supply tube 16. Optionally, additional couplers 18 can be forcibly inserted over the endoscopic insertion unit 68 and the inflation fluid supply tube 16 perpendicular to the longitudinal axis 80 as appropriate to selectively couple and engage the endoscope 20 and the balloon dilation device 11. More than one coupler 18 can be used as appropriate for the length and desired securement of the system 10.

The inflation fluid supply tube 16 can be longitudinally fixed with respect to the endoscopic insertion unit 68. In the assembled state, the viewing end 72 of the endoscope 20 can be positioned proximal to the first end 38 of the inflation fluid supply tube 16. In one embodiment, the viewing end 72 of the endoscope 20 can be spaced a distance “d” from the first end 38 of the inflation fluid supply tube 16, and thus, also spaced from the balloon 14. The distance “d” is suitable to allow the balloon 14 to be inflated without interference, or obstruction, from the endoscope 20. In one embodiment, the distance “d” between the viewing end 72 and the first end 38 of the inflation fluid supply tube 16 can be 1 centimeter (cm). The system 10 can allow the surgeon to bend the distal portion 22 of the inner member 12 to accommodate the patient's anatomical constraints and to position the balloon 14 at least partially out of the field of view of the endoscope 20 for visualization. In some embodiments, the distal portion 22 of the inner member 12 maintaining the balloon 14 can be curved transversely away from the viewing end 72 of endoscope 20.

The dilation treatment system 10 formed of the combined endoscope 20 and balloon dilation device 11 allows the surgeon to use a single hand to manipulate the system 10 within the patient's anatomy to simultaneously visualize and treat, for example, the patient's Eustachian tube. In inserting the treatment system 10 into the patient's anatomy, the endoscope 20 is inserted and advanced simultaneously with balloon dilation device 11 for the treatment and observation of the Eustachian tube. In particular, the position of the distal portion 22 of the inner member 12 within the patient's anatomy can be visualized through the viewing end 72 of the endoscope 20 as the dilation treatment system 10 is advanced or otherwise positioned within the patient's anatomy. The distal portion 22 of the inner member 12 is maneuvered and positioned at a target site within the patient's anatomy with the assistance of the visualization provided by the endoscope 20.

During use, the second end 40 of the inflation fluid supply tube 16 can be fluidly coupled to an inflation source (not shown). The inflation source supplies pressurized fluid to inflate the balloon 14 through the inflation fluid supply tube 16 when the balloon 14 positioned at the target site, as confirmed by visualizing the position with the endoscope 20. The lumen 32 of the inner member 12 can be used for pressure relief of the patient's anatomy in response to, and in some cases simultaneously to, inflation of the balloon 14. Bodily fluids can be vented, suctioned, or otherwise removed, from the patient's anatomy through the lumen 32 of the inner member 12 during insertion as well as during and after inflation, for example. The balloon 14 may be held in the target position while maintained in the expanded state for an extended period of time, for example, several seconds or minutes. The extended period of time is suitable to effect an open dilated state to the Eustachian tube of the patient.

The balloon 14 may be returned to a deflated state and repositioned by advancing or withdrawing the dilation treatment system 10 within the patient's anatomy with the endoscope 20 providing visualization to properly reposition in the desired location within the Eustachian tube. The balloon 14 can then again be inflated and maintained in the expanded state for a suitable extended period of time to effect an open dilated state of the desired anatomical passageway. The balloon 14 can be deflated and the dilation treatment system 10 can be withdrawn from the patient's anatomy in a direction reverse of the insertion and advancement. In some embodiments, once withdrawn, the coupler 18 can be removed to uncouple and release the balloon dilation device 11 from the endoscope 20. The balloon dilation device 11 and the endoscope 20 can then independently be sterilized or disposed of.

Although the present disclosure has been described with reference to preferred embodiments, workers skilled in the art will recognize that changes can be made in form and detail without departing from the spirit and scope of the present disclosure.

What is claimed is:

1. A device for use in dilating an Eustachian tube, the device comprising:
an inner member comprising an elongated tubular body and a lumen fluidly open at a distal end and a proximal end, the inner member having a distal portion terminating at the distal end, a proximal portion terminating at the proximal end, and a central portion extending between the distal portion and the proximal portion;
an inflation fluid supply tube fixedly disposed around the central portion of the inner member and including an inflation lumen, the inflation fluid supply tube having a first end, a second end opposite the first end, and an opening disposed between the first end and the second end, wherein the proximal portion of the inner member extends through the opening and the opening is fluidly sealed against an exterior surface of the inner member, and wherein the distal portion of the inner member extends distally beyond the first end;
a balloon having a distal side opposite a proximal side, wherein the distal side is coupled to the inner member adjacent the distal end of the inner member and the proximal side of the balloon is in fluid communication with the inflation lumen; and
a coupler configured to selectively couple the inflation fluid supply tube to an endoscope, wherein the coupler is configured to engage against an exterior surface of the inflation fluid supply tube and be disposable between the first end and the opening of the inflation fluid supply tube.

2. The device of claim 1, wherein the coupler is configured to frictionally engage with an outer surface of an endoscope sheath disposed along the endoscope.

3. The device of claim 1, wherein the inner member and the inflation fluid supply tube are each formed of a malleable material.

4. The device of claim 1, wherein the proximal end of the inner member is disposed exterior of the inflation fluid supply tube.

5. The device of claim 1, wherein the coupler longitudinally fixes the inflation fluid supply tube to the endoscope.

6. The device of claim 1, wherein the device includes at least two of the couplers spaced from one another along a length of the inflation fluid supply tube.

7. A dilation treatment system for use in dilating an Eustachian tube of a patient the system comprising:
a dilation treatment device comprising:
an inner member comprising an elongated tubular body and a lumen, the lumen fluidly open at and between a distal end and a proximal end;
an inflation fluid supply tube fixedly disposed around a central portion of the inner member, the inflation fluid supply tube defining a first end, a second end, and an inflation lumen extending between the first end and the second end, the distal end of the inner member extended distally of the first end, the proximal end of the inner member extended through an opening of the inflation fluid supply tube;
a balloon mounted to a distal portion of the inner member and fluidly coupled to the inflation lumen;
an endoscope including an endoscopic insertion unit defining a viewing end; and
a coupler configured to selectively couple the endoscopic insertion unit to the dilation treatment device with the endoscopic insertion unit extending parallel and longitudinally fixed relative to the inflation fluid supply tube, wherein the viewing end is disposed alongside a distal section of the inflation fluid supply tube, wherein the coupler is disposed between the first end and the opening of the inflation fluid supply tube.

8. The system of claim 7, wherein the distal portion of the inner member extends transversely away from the viewing end.

9. The system of claim 7, wherein the endoscopic insertion unit includes a sheath disposed on an insertion body, and further wherein the inner member and the endoscopic insertion unit are selectively coupled in parallel along an insertion length of the sheath.

10. The system of claim 7, wherein the viewing end is proximal the balloon.

11. The system of claim 7, wherein the balloon is disposed distal the viewing end.

12. The system of claim 7, wherein the inflation fluid supply tube is configured to maintain a pre-determined length during pressurization of the balloon.

13. The system of claim 7, wherein the coupler frictionally engages the endoscopic insertion unit and the inflation fluid supply tube.

14. The system of claim 7, wherein the coupler fixedly maintains the inflation fluid supply tube relative to the endoscopic insertion unit.

15. The system of claim 7, wherein the coupler releasably couples the endoscopic insertion unit and the dilation treatment device.

16. The system of claim 7, wherein the endoscopic insertion unit includes an insertion body disposed within a sheath, and further wherein the coupler is disposed around a circumference of the sheath and of the inflation fluid supply tube.

17. A method of endoscopically treating an Eustachian tube of a patient, comprising:
coupling a dilation treatment device and an endoscope together with one or more couplers to form a dilation treatment system, wherein the endoscope includes an endoscopic insertion unit defining a viewing end, and further wherein the dilation device comprises:
an inner member fixedly extending within an inflation fluid supply tube, the inner member including a distal portion extending distal a first end of the inflation fluid supply tube, a proximal portion extending proximally outside of the inflation fluid supply tube, and a lumen fluidly open at a distal end and a proximal end, and
a balloon disposed on the distal portion of the inner member and fluidly coupled to the inflation fluid supply tube;
wherein the step of coupling includes the dilation treatment device and the endoscopic insertion unit longitudinally fixedly aligned and coupled together by the one or more couplers, and the viewing end fixedly disposed proximal to the first end of the inflation fluid supply tube and proximal to the distal portion;
advancing the dilation treatment system into a patient's anatomy;
visualizing a position of the distal portion of the inner member within the patient's anatomy with the endoscope;
positioning the distal portion of the inner member at a target site within the patient's Eustachian tube;
inflating the balloon through the inflation fluid supply tube at the target site; and
dilating an opening of the patient's Eustachian tube at the target site with the inflated balloon.

18. The method of claim 17, further comprising:
suctioning bodily fluids from the patient's anatomy through the lumen of the inner member.

19. The method of claim 18, wherein the steps of suctioning and inflating are performed simultaneously.

20. The method of claim 17, wherein the steps of visualizing and positioning are performed simultaneously.
